(19) United States
(12) Patent Application Publication
(10) Pub. No.: US 2018/0185050 A1
(43) Pub. Date: Jul. 5, 2018

(54) DISSECTING PUNCH FOR FOLLICULAR EXTRACTION AND TOOLS AND METHODS USING SAME

(71) Applicant: HSC Development, LLC, Greenwood Village, CO (US)

(72) Inventor: James A. Harris, Denver, CO (US)

(21) Appl. No.: 15/676,556

(22) Filed: Aug. 14, 2017

Related U.S. Application Data
(63) Continuation of application No. 13/939,909, filed on Jul. 11, 2013, now abandoned.

(51) Int. Cl. A61B 17/3205; A61B 17/00

(52) U.S. Cl. CPC A61B 17/32053; A61B 2017/00752

(57) ABSTRACT

A dissecting punch and tool configured for the dissection of follicular units from the donor area of a patient, such as for subsequent implantation of a follicular unit graft (FUG) in a recipient area for hair restoration, and method for using the tool. The method includes aligning the dissecting punch over a follicular unit so that hair follicles are disposed within the lumen. The dissecting punch is then moved through the dermis layer and into the fatty tissue layer of the skin to dissect the follicular unit from the tissue surrounding the follicular unit. The dissecting punch includes a non-circular shaft segment to reduce the transection rate of the follicular units during dissection.

### CROSS-REFERENCE TO RELATED APPLICATIONS

This application claims priority as a continuation application of U.S. patent application Ser. No. 13/939,909, entitled DISSECTING PUNCH FOR FOLLICULAR EXTRACTION AND TOOLS AND METHODS USING SAME filed on Jul. 11, 2013, the disclosure of which is incorporated herein by reference in its entirety.

### BACKGROUND

### 1. Field

This disclosure relates to tools and methods for the extraction of hair follicles from the donor area of a patient for subsequent transplantation to a recipient area. More specifically, the present invention relates to a dissecting punch that is configured for the dissection of hair follicles, and tools and methods implementing the dissecting punch that may decrease the follicular transection rate and may improve the yield of transplantable hair follicles as compared to known follicular extraction tools and methods.

### 2. Related Art

The basic process of hair transplantation includes removing (e.g., extracting) hair follicles from donor areas (e.g., the side and back fringe areas of the patient's head), and implanting the follicles into recipient areas (e.g., bald areas). Historically, 4 mm diameter plugs were utilized as the donor plug; this was followed by mini-grafts (smaller plugs), and finally by follicular units grafts (FUG's). Follicular units are naturally occurring follicle aggregates (e.g., 3 to 5 closely spaced hair follicles) that are distributed randomly over the surface of the scalp.

In the foregoing processes, a linear portion of the scalp is removed from the donor area by dissection with a scalpel forming a donor strip. Some follicles are invariably transected during this process, damaging the follicles such that they cannot be transplanted. In addition to some follicular damage, the removal of this donor strip will always result in a scar. In addition to the scar, there is usually a degree of moderate pain for several days and a sensation of tightness for 6 to 8 weeks following the procedure. Multiple procedures will result in multiple scars and thinning of the hair above and below the scar. If the pliability or laxity of the scalp is miscalculated, and a strip that is too wide is removed, this procedure has the potential to create a wide, unsightly scar because of the tension required to close the wound. Sometimes the resulting scar can be difficult to hide or disguise, causing a significant cosmetic deformity.

FUG's are dissected from a donor strip by several technicians using operating microscopes. Often the best technicians can produce approximately 250 to 300 FUG's per hour, and an average technician produces closer to 200 FUG's per hour. The FUG's are sorted into groups based upon the number of hair follicles contained in the FUG. The best technicians will have a transection rate of from about 2% to 5%.

In about 2002, Rassman et al. disclosed a technique called follicular unit extraction (FUE), whereby follicular units were extracted from the donor area without the need to create a linear incision with a scalpel. This was accomplished by using a sharp 1 mm diameter punch to make an incision into the epidermis and dermis, and then removing the follicular unit from the surrounding skin with forceps. Their findings suggested that some follicles were easily removed, while others had a significant tendency to shear in the process. By their research, a good candidate was defined as one who experienced less than 20% shearing, and only about 25% of the patients tested were considered good candidates by their shearing test. This test is called the FOX (FOllicular eXtraction) test.

The foregoing procedure is technically difficult, as the penetration depth and penetration angle is difficult to control. If the sharp punch penetrates too deeply or at the incorrect angle there is a good chance of transecting the follicular unit. This method has not been widely adopted due to the problems of transection, difficulty removing the grafts, the time required to produce the grafts, and the low percentage of potential candidates

Dr. John Cole subsequently devised a device that limits the depth of the sharp punch to just below the attachment of the arrector pili muscle, which is presumably responsible for tethering the follicular unit to the deeper tissues, and which caused shearing during extraction with forceps. He has called his procedure the FIT, or Follicular Isolation Technique.

The dissection of grafts from the scalps of African Americans and those with a high percentage of gray or white hairs is particularly problematic. The African American's follicles typically have a high degree of curl or curve, making the dissection difficult and prone to high transection rates. The follicles of white or gray hair are all but invisible, even under the microscope, making them prone to a high rate of transection as well.

There remains a need for a follicular extraction method and related devices that reduce the amount of follicular transection and increases the follicular extraction rate.

### SUMMARY

The present disclosure relates to a dissecting punch for the dissection of follicular units from the donor area of a patient, such as for the formation of a follicular unit graft (FUG) for subsequent implantation in a recipient area. The dissecting punch is inserted into the skin over the follicular unit and is rotated (e.g., manually or mechanically) during the dissection. At least a portion of the dissecting punch has a non-circular (e.g., polygonal) cross-section, which causes the tissue surrounding the follicular unit to oscillate, e.g., to move in a direction that is transverse to the dissecting punch, when the dissecting punch is rotated during the dissection of the follicular unit. It has been unexpectedly found that this oscillation of the surrounding tissue decreases the transection rate of the follicular units as compared to other known tools and methods for follicular unit extraction.

In one embodiment, a dissecting punch that is configured for dissecting follicular units from the skin surface of a donor is provided. The dissecting punch may include a dissecting punch body and a lumen that is axially disposed through at least a portion of the dissecting punch body. The punch body includes a dissecting shaft segment, where the shaft segment includes an outer surface defining a non-circular cross-section. The punch body also includes a dissecting tip segment that is disposed at a distal end of the shaft segment. The dissecting tip segment includes an outer surface extending from the shaft segment to a distal dissecting edge that circumvents a distal end of the lumen. As is noted above, the non-circular cross-section of the shaft segment may advantageously reduce transection rates of follicular units during dissection.

In one characterization, the distal dissecting edge is substantially circular. For example, the distal dissecting edge may have internal diameter of at least about 0.5 mm and not greater than about 2.0 mm. The dissecting tip segment may have a length of at least about 0.5 mm and not greater than about 2.0 mm. In another characterization, the diameter of the dissecting tip segment outer surface may be tapered inwardly from proximal (e.g., adjacent to) the shaft segment to the distal dissecting edge to facilitate insertion of the tip segment into the skin surface.

As is noted above, the dissecting shaft segment comprises an outer surface defining a non-circular cross-section. In one characterization, the shaft segment outer surface includes at least a first longitudinally-extending projection (e.g., a rib). For example, the shaft segment outer surface may include a plurality of longitudinally-extending projections. In another characterization, the shaft segment outer surface defines a polygonal cross-section. For example, the shaft segment outer surface may define a hexagonal cross-section, or a pentagonal cross-section.

In any event, the shaft segment may have a length of at least about 2.0 mm and not greater than about 6.0 mm. Further, the shaft segment may have an effective outer diameter of at least about 1.0 mm and not greater than about 1.7 mm.

In another characterization, the lumen extends through the shaft segment, e.g., completely through the shaft segment. In another characterization, the dissecting punch body may be fabricated from a metal such as stainless steel. In another characterization, the dissecting punch may include a shoulder at a proximal end of the shaft segment, where the shoulder has an outer diameter greater than an effective outer diameter of the shaft segment. The shoulder may function as a depth-limiting mechanism. For example, the shoulder may be defined by a shoulder block disposed at a proximal end of the punch body. In one characterization, a gripping shaft may extend from a proximal end of the shoulder block. The punch body, shoulder block and gripping shaft may be integrally formed, e.g., as a single monolithic body.

The distal dissecting edge may be characterized as being blunt, e.g., not sharpened. For example, the distal dissecting edge may have a small radius of curvature, such as a radius of curvature of at least 0.025 mm. In another characterization, the distal dissecting edge may be substantially flat.

In one particular characterization, a dissecting punch that is configured for dissecting a follicular unit from the skin surface of a donor includes a dissecting punch body and a lumen that is axially disposed through at least a portion of the dissecting punch body. The punch body includes a dissecting shaft segment, where the shaft segment includes an outer surface defining a polygonal cross-section. The punch body further includes a dissecting tip segment disposed at a distal end of the shaft segment. The dissecting tip segment includes an outer surface extending from the shaft segment to a distal dissecting edge circumventing a distal end of the lumen, wherein a diameter of the dissecting tip segment outer surface is tapered inwardly from the shaft segment to the distal dissecting edge to facilitate insertion of the tip segment into the skin surface. The polygonal cross-section may be a hexagonal cross-section, for example.

In another embodiment, a tool that is configured for dissecting follicular units from the skin surface of a donor is provided. The tool includes a sharp scoring punch and a dissecting punch body in a lumen that is axially disposed through at least a portion of the dissecting punch body. The dissecting punch body includes a dissecting shaft segment, the shaft segment including an outer surface defining a non-circular cross-section, and a dissecting tip segment disposed at a distal end of the shaft segment. The dissecting tip segment includes an outer surface extending from the shaft segment to a distal dissecting edge circumventing a distal end of the lumen. The sharp scoring punch includes a sharp cutting edge that is sharper than the distal dissecting edge.

In one characterization, the distal dissecting edge is substantially circular, and the distal dissecting edge may have an internal diameter of at least about 0.5 mm and not greater than about 2.0 mm.

As is noted, the dissecting shaft segment defines a non-circular cross-section. In one characterization, the shaft segment outer surface includes at least a first longitudinally-extending projection, and may include a plurality of longitudinally-extending projections. In another characterization, the shaft segment outer surface defines a polygonal cross-section, such as a hexagonal cross-section. The shaft segment may have a length of at least about 2.0 mm and not greater than about 6.0 mm.

The distal dissecting edge may be characterized as being blunt, e.g., not sharpened. In one characterization, the distal dissecting edge has a radius of curvature of at least about 0.025 mm. In another characterization, the distal dissecting edge is substantially flat.

The sharp scoring punch may be operatively attached to the dissecting punch body, and in one characterization the sharp scoring punch is disposed within the lumen that is axially disposed through the dissecting punch body. For example, the sharp scoring punch may be retractable within the lumen.

In another embodiment, a tool that is configured for dissecting follicular units from the skin surface of a donor is provided. The tool may include a dissecting punch body and a lumen that is axially disposed through at least a portion of the dissecting punch body. The punch body includes a dissecting shaft segment, where the shaft segment includes an outer surface defining a non-circular cross-section. The dissecting punch body also includes a dissecting tip segment that is disposed at a distal end of the shaft segment, where the dissecting tip segment includes an outer surface extending from the shaft segment to a distal dissecting edge circumventing a distal end of the lumen. A rotation mechanism is operatively coupled to the dissecting punch body and is configured to rotate the dissecting punch body about a longitudinal axis thereof.

In one characterization, the distal dissecting edge is substantially circular, and may have an internal diameter of at least about 0.5 mm and not greater than about 2.0 mm.

In one characterization, the shaft segment outer surface includes at least a first longitudinally-extending projection, and may include a plurality of longitudinally-extending projections. In another characterization, the shaft segment outer surface defines a polygonal cross-section, such as a hexagonal cross-section. The shaft segment may have a length of at least about 2.0 mm and not greater than about 6.0 mm.

In another characterization, the tool further includes a shoulder at proximal end of the shaft segment, where the shoulder has an outer diameter greater than an effective outer diameter of the shaft segment. The shoulder may be defined by a shoulder block disposed at a proximal end of the punch body.

The distal dissecting edge may be characterized as being blunt, e.g. not sharpened. In one characterization, the distal dissecting edge has a radius of curvature of at least about 0.025 mm. In another characterization, the distal dissecting edge is substantially flat.

In another characterization, the rotation mechanism is configured to rotate the dissecting punch body at a rotational speed of at least about 2000 rpm. For example, the rotation mechanism may be a drill wherein the dissecting punch body is operatively attached to the drill, for example, via a chuck. Further, the dissecting punch body may be operatively fixed to a computer-controlled robotic arm for automated use of the dissecting punch body.

In another embodiment, a method for the dissection of a follicular unit from the skin of a donor is provided. The method may include the step of placing a dissecting punch body having a lumen therethrough over a follicular unit. The dissecting punch body is rotated and is moved into the skin whereby the follicular unit is disposed within the lumen. The dissecting punch body may include a dissecting shaft segment, the shaft segment including an outer surface defining a non-circular-section, and a dissecting tip segment that is disposed at a distal end of the shaft segment. The dissecting tip segment may include an outer surface extending from the shaft segment to a distal dissecting edge circumventing a distal end of the lumen.

Rotation of the dissecting punch body, including the shaft segment into the skin causes the tissue surrounding the follicular unit to oscillate during rotation of the dissecting punch body to advantageously decrease the transaction rate of follicular units.

In one characterization, the method includes the step of scoring the skin over the follicular unit with a sharp scoring punch before moving the rotating dissecting punch body into the skin. For example, the sharp scoring punch may be retractable within the lumen of the dissecting punch body. In another characterization, the dissecting punch body is rotated at a rotational speed of at least about 3000 rpm during the moving step, such as at least about 3500 rpm. The dissecting punch body may be a dissecting punch body according to any of the foregoing embodiments and characterizations.

In addition to the exemplary embodiments and characterizations described above, further embodiments and characterizations will become apparent by reference to the drawings and the following description.

### DESCRIPTION OF THE DRAWINGS

FIGS. 1A to 1F schematically illustrate a follicular extraction method.

FIGS. 2A to 2F schematically illustrate an alternative follicular extraction method.

FIG. 3 illustrates a perspective view of a dissecting punch that is configured for follicular extraction.

FIG. 4 illustrates an end view of the dissecting punch that is configured for follicular extraction illustrated in FIG. 3.

FIG. 5A illustrates a further cross-sectional view of the dissecting punch for follicular extraction illustrated in FIG. 3.

FIG. 5B illustrates a cross-sectional view of an alternative embodiment of a dissecting punch that is configured for follicular extraction.

FIG. 6 illustrates a perspective view of a dissecting punch that is configured for follicular extraction.

FIG. 7 illustrates a cross-sectional view of the dissecting punch for follicular extraction illustrated in FIG. 6.

FIG. 8 illustrates a perspective view of a dissecting punch that is configured for follicular extraction.

FIG. 9 illustrates a cross-sectional view of the dissecting punch for follicular extraction illustrated in FIG. 8.

FIG. 10 illustrates a perspective view of a dissecting punch.

FIG. 11 illustrates a perspective view of powered tool including a dissecting punch that is configured for follicular-extraction.

FIG. 12 illustrates a perspective view of a tool including a dissecting punch that is configured for follicular extraction.

### DESCRIPTION

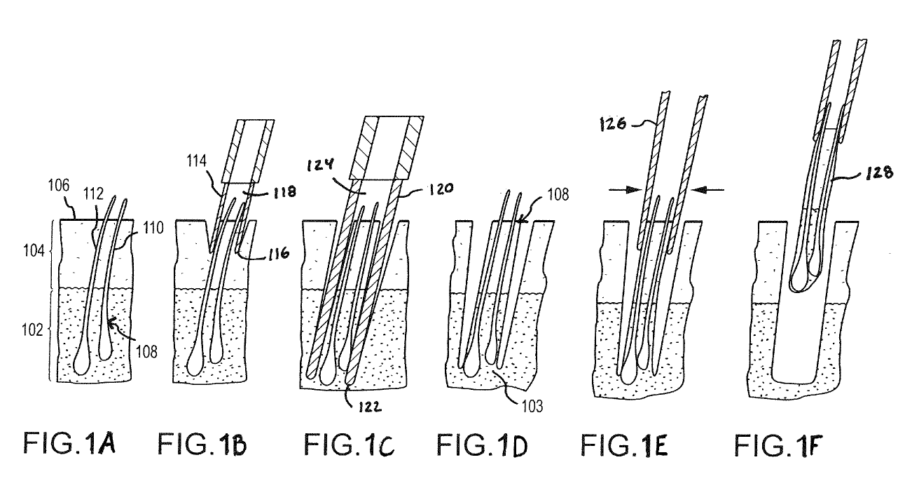

FIGS. 1A to 1F schematically illustrate a follicular extraction device and method according to U.S. Pat. No. 8,211,117 by Harris, which is incorporated herein by reference in its entirety. Generally, the method illustrated in FIGS. 1A to 1F includes a two-step technique for the dissection of the follicular unit from surrounding tissue for subsequent extraction from the donor area to form a follicular unit graft (FUG). The first step scores the skin surrounding the follicular unit with a sharp scoring punch. The second step separates the follicular unit from the surrounding tissue and fat with a blunt dissecting punch to reduce the risk of shearing the hair follicles.

Referring to FIGS. 1A to 1F, the patient's skin (e.g., a donor area) includes a fatty layer 102, dermis 104, and epidermis 106. A follicular unit 108 is anchored in the fatty layer 102. The illustrated follicular unit 108 consists of two hair follicles, 110 and 112, extending through the dermis 104 and epidermis 106.

The method illustrated in FIGS. 1A to 1F includes the use of a sharp scoring punch 114 (FIG. 1B)). The sharp scoring punch 114 includes a sharp cutting edge 116 that may be circular in cross-section and may have an inner diameter of at least about 0.1 mm and not greater than about 1.1 mm, for example. The sharp cutting edge 116 of the scoring punch 114 has a sufficient sharpness to cut through the epidermis 206, dermis 204 and fatty layer 202 with relative ease, as is known to those skilled in the art. An example of such a sharp scoring punch is the 1 mm Dermal Biopsy Punch available from Miltex, Inc., Bethpage, N.Y.

The sharp scoring punch 114 is aligned approximately parallel to the portion of the hairs 110 and 112 protruding from the epidermis 106 with the protruding hair portions being disposed within a lumen 118 of the scoring punch 114. A limited amount of force is then applied to the scoring punch 114, possibly including rotation, such that the cutting edge 116 cuts through the epidermis 106 and enters the dermis 104. The sharp scoring punch 114 is inserted to a depth that is sufficient to score the epidermis 106 and dermis 104, but that is not so deep as to risk transection of the hair follicles 110 and 112. For example, the sharp cutting edge 116 may be inserted to a total depth of not greater than about 1.5 mm. The sharp scoring punch 114 may then be removed.

As is illustrated by FIG. 1C, a blunt dissecting punch 120 is then placed into the incision created by the scoring punch 114. The blunt dissecting punch 120 has an inner diameter that is fractionally larger than the outer diameter of the scoring punch 114, whereby the dissecting punch 120 can readily advance through the incision created by the scoring punch 114. The blunt dissecting punch 120 is less sharp than the scoring punch 114, and the blunt dissecting edge 122 of the dissecting punch 120 is configured such that the probability of shearing a hair follicle (e.g., hair follicle 110) is very low. However, the dissecting punch 120 is capable of advancing through the softer dermis 104 and into the fatty layer 102 without extraordinary pressure (force) being applied to the dissecting punch 120.

The blunt dissecting edge 122 is advanced through the dermis 104 and into the fatty layer 102 to a depth that is sufficient to enable the subsequent removal of the follicular unit 108 in the form of a follicular unit graft 128 (i.e., the follicular unit 108 and immediate surrounding tissue) without substantially damaging the follicular unit 108. Accordingly, the dissecting punch 120 (e.g., the blunt dissecting edge 122) penetrates to a depth that is deeper than the insertion depth of the scoring punch 114 (e.g., the cutting edge 116) and can be fully inserted through the dermis 104 and into the fatty layer 102. The dissecting punch 120 may be inserted to a total depth of at least about 1.5 mm and not greater than about 8 mm, for example. The blunt dissecting punch 120 bluntly separates (e.g., dissects) the fibrous attachments surrounding the follicular unit 108, leaving it attached only at its base 103 (FIG. 1D)). The follicular unit 108 may then be easily removed from the skin using forceps 126, or a similar device. The follicular unit 108 is removed intact as a FUG 128 and is ready for implantation at a recipient site.

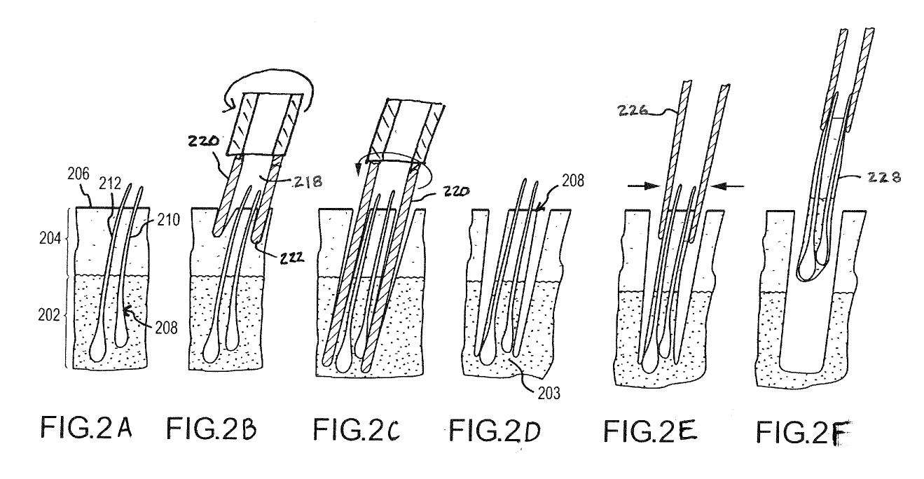

FIGS. 2A to 2F schematically illustrate a device and method that is disclosed in U.S. Patent Publication No. 2010/0114118 by Harris, which is incorporated herein by reference in its entirety. As is described above with respect to FIGS. 1A to 1F, the donor's skin includes a fatty layer 202, dermis 204 and epidermis 206. A follicular unit 208 is embedded in the skin and includes two hair follicles 210 and 212 that extend through the dermis and epidermis and are anchored in the fatty layer 202.

In contrast to the method illustrated in FIGS. 1A to 1F, the method illustrated in FIGS. 2A to 2F includes the use of a single blunt dissecting punch 220 that is rapidly rotated to dissect the follicular unit 208. Specifically, the blunt dissecting punch 220 having a blunt dissecting edge 222 may be aligned over the hair follicles 210 and 212 and may be rotated at a sufficiently high rotational speed to enable the blunt dissecting edge 222 of the blunt dissecting punch 220 to score (e.g., cut through) the epidermis 206 without undo pressure being applied to the blunt dissecting punch 220 (FIG. 2B)). As is illustrated in FIG. 2C, the blunt dissecting punch 220 (e.g., the blunt dissecting edge 222) may be moved through the dermis 204 and fatty layer 202 while continuing to rotate. The rotational speed of the blunt dissecting punch 220 may be decreased as the blunt dissecting edge 222 moves through the dermis 204 and into the fatty layer 202. As a result, the rotational speed as the blunt dissecting edge 222 moves through the fatty layer 202 may be low enough so that the probability of dissecting the follicular unit 208 is low, while being high enough that the dissecting punch 220 can move through the fatty layer 202 without extraordinary pressure being applied to the dissecting punch 220.

As illustrated in FIG. 2D, the blunt dissecting punch 220 may then be removed leaving the follicular unit 208 loosely attached at its base 203. Forceps 226 or a similar device may then be utilized to remove the follicular unit 208 from the skin, in the form of a FUG 228. See FIG. 2E and FIG. 2F.

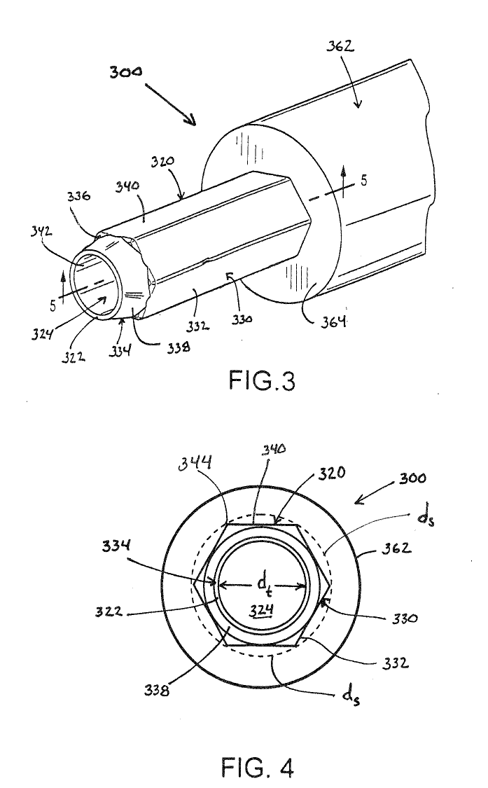

FIG. 3 illustrates a perspective view of a dissecting punch that is configured for dissecting follicular units from the skin surface of a patient in accordance with one embodiment of the present disclosure. The dissecting punch 300 includes a dissecting punch body 320 and a lumen 324 that is axially disposed through at least a portion of the dissecting punch body 320.

The punch body 320 includes a dissecting shaft segment 330 having an outer surface 332 that defines a non-circular cross-section. That is, the outer surface 332 is non-circular around its outer circumference. As illustrated in FIG. 3, the outer surface 332 defines a polygonal cross-section, namely a hexagonal (e.g., 6-sided) cross-section. In another characterization, the outer surface 332 comprises a plurality of distinct and substantially planar surfaces (e.g., planar surface 340) that extend along the length of the shaft segment 330 and that intersect adjacent planar surfaces on the circumference of the outer surface 332. In this characterization, the outer surface 332 may include 3 or more distinct planar surfaces, such as 4, 5, 6, 7, 8, 9, 10 or more planar surfaces.

A dissecting tip segment 334 is disposed at a distal end 336 of the dissecting shaft segment 330. The dissecting tip segment 334 includes an outer surface 338 that extends from the dissecting shaft segment 330 to a distal dissecting edge 322 that circumvents a distal end 342 of the lumen 324. The dissecting tip segment 334 may have a size (e.g., an outer diameter) at the distal end 336 of the shaft segment that is less than the outer diameter (e.g., the effective outer diameter) of the dissecting shaft segment 330.

In one characterization, the distal dissecting edge 322 is substantially circular to facilitate the initial penetration of the skin (e.g., the epidermis) by the punch body 320 as the punch body 320 is rotated while in contact with the skin. To further facilitate the insertion of the dissecting tip segment 334 into the skin surface, at least a portion of the dissecting tip segment outer surface 338 may be tapered inwardly (e.g., may decrease in diameter) from proximal the shaft segment 330 (e.g., from a shaft segment distal end 336) to the distal dissecting edge 322. Stated another way, at least a portion of the dissecting tip segment 334 may be frustoconical in shape. In this manner, as the rotating dissecting tip segment 334 enters the skin surface, the tapered outer surface 338 will slightly push the outer layer of skin away from the follicular unit and facilitate the movement of the dissecting shaft segment 330 into the incision created by the distal dissecting edge 322. In one characterization, the dissecting shaft segment 330 is tapered along substantially its entire length.

The dissecting punch 300 illustrated in FIG. 3 also includes a shoulder block 362 disposed at a proximal end of the dissecting punch body 320. As illustrated in FIG. 3, the shoulder block 362 is substantially cylindrical in shape and extends axially away from the dissecting punch body 320. The shoulder block 362 includes a distal end defining a shoulder 364. The shoulder 364 may function as a depth-limiting mechanism to limit the depth of insertion of the dissecting punch body 320, e.g., to the length of the shaft segment 330 and the dissecting tip segment 334. In this regard, the shoulder may have an outer diameter (e.g., a circumference) that is larger than the diameter of the shaft segment 330. The shoulder block 362 may also facilitate handling of the tool 300, such as when placing the tool 300 into a chuck of a drill or similar device, as is described below. Although illustrated as a cylindrical body defining a circular shoulder 364, the shoulder block may have any configuration such as a polygonal or oval cross-section defining a non-circular shoulder. The lumen 324 may extend partially through the dissecting punch body 320 or may extend through the entire punch body 320. Further, the lumen 324 may extend through the shoulder block 362, such as when it is desirable to attach a suction device to the tool to remove follicular unit grafts from the lumen 324.

FIG. 4 illustrates an end view of the dissecting punch 300 illustrated in FIG. 3. As is illustrated in FIG. 4, the shaft segment 330 includes an outer surface 332 having a non-circular cross-section, where the cross-section is polygonal, and more specifically is hexagonal. Although the corners of the polygon (e.g., corner 344) are illustrated as being angled, the corners of the polygon may also be rounded, such as to reduce frictional forces when the rotating shaft segment 330 is inserted into the skin. In other embodiments, the cross-section of the shaft segment outer surface 332 may comprise other polygonal shapes or other non-circular shapes.

The dissecting distal edge 322 may have an internal diameter (dₜ) that is sized to facilitate the dissection of follicular units. For example, the diameter dₜ may be at least about 0.1 mm, such as at least about 0.5 mm, or at least about 0.7 mm. Further, the internal diameter dₜ may be not greater than about 2.0 mm, such as not greater than about 1.5 mm, or not greater than about 1.0 mm for example. The lumen 324 may have a substantially uniform diameter throughout its length, such that the diameter of the lumen 324 at its distal end 342 (i.e., the internal diameter dₜ of the dissecting distal edge 322) is substantially the same along the length of the lumen 324. Alternatively, the lumen 324 may have a diameter that varies through its length, such as to accommodate the removal of follicular unit grafts from the lumen by using suction (e.g., a vacuum).

The shaft segment 330 is also sized to accommodate the dissection of follicular units from the skin of a patient, without causing significant trauma to the skin surrounding the follicular unit. In this regard, the shaft segment 330 may have an effective shaft diameter (dₛ) that facilitates the dissection of the follicular units in this manner. As used herein, the effective shaft diameter dₛ is the diameter of the smallest circle that encompasses all of the points on the outer surface 332 of the shaft segment 330. See FIG. 4. The effective outer diameter dₛ may be at least about 0.8 mm, such as at least about 1.0 mm or at least about 1.1 mm. To reduce the potential of trauma to the surrounding tissue, the effective diameter dₛ may be not greater than about 2.0 mm, such as not greater than about 1.7 mm. In another characterization, the effective outer diameter is at least about 0.1 mm, such as at least about 0.2 mm or at least about 0.3 mm greater than the outer diameter of the tip segment 334 adjacent to the distal end 336 of the shaft segment.

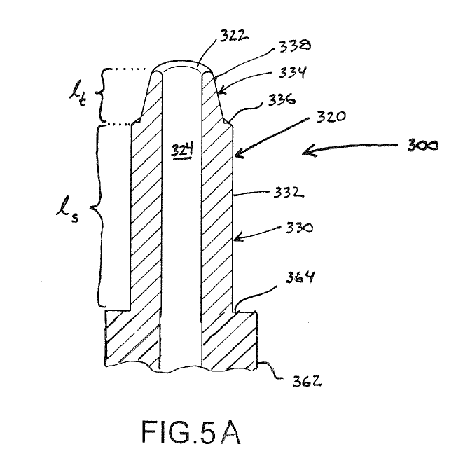

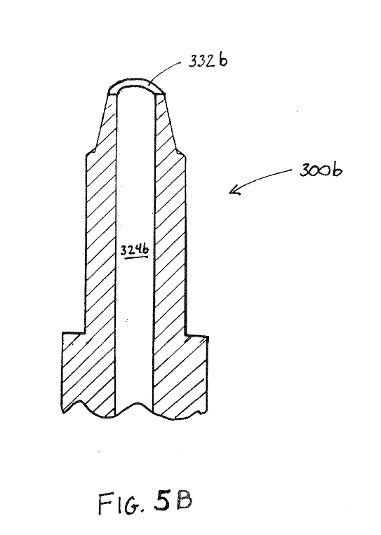

FIG. 5A illustrates a cross-sectional view of the dissecting punch 300. As is illustrated in FIG. 5A, the dissecting tip segment 334 is disposed at the distal end 336 of the dissecting shaft segment 330 and includes an outer surface 338. The outer surface 338 is tapered inwardly from proximal the shaft segment 330 to a distal dissecting edge 322 to facilitate insertion of the punch body 320 into the skin. The dissecting punch 300 also includes a shoulder body 362 defining a shoulder 364 that is sized and configured to limit the depth of insertion of the dissecting punch 300 into the skin during dissection. The lumen 324 is disposed through the punch body 320, and is also disposed through the shoulder body 362.

The punch body 320 is configured and sized to be useful for the dissection of follicular units from the skin of a patient. In this regard, the dissecting tip segment 334 may have a length (It) of at least about 0.5 mm, such as at least about 0.75 mm. Further, the length lₜ may be not greater than about 2.5 mm, such as not greater than about 2 mm, such as not greater than about 1.5 mm. The shaft segment 330 may have a length (Is) of at least about 2 mm, such as at least about 3.5. The length Is may be not greater than about 6 mm, such as not greater than about 4.5 mm. A proximal end of the shaft segment 330 terminates at the shoulder 364. The shoulder 364 may be of sufficient size (e.g., width) to serve as a depth-limiting mechanism, limiting the depth of insertion of the punch body 320 into the skin.

The dissecting distal edge 322 may be characterized as being blunt, e.g., not sharp. That is, the dissecting distal edge 322 may be characterized as being sufficiently thin to enable the dissecting distal edge 322 to pass through the epidermis when the punch body 320 is rotated with sufficient rotational speed, and sufficiently thin to pass through the dermis and into the fatty layer of the skin when rotated at slower speeds. However, the dissecting distal edge 322 is sufficiently dull (e.g., not sharpened) such that the probability of dissecting a follicular unit is low. That is, rather than transect a follicular unit if the distal edge is pushed against the follicular unit, the blunt dissecting edge 322 will tend to push the follicular unit into the lumen 324. In this regard, the dissecting distal edge 322 may be slightly rounded as illustrated in FIG. 5a, for example such that the distal edge 322 forms a truncated torus. In one characterization, the rounded dissecting distal edge 322 may have a radius of curvature of at least about 0.025 mm and not greater than about 0.075 mm.

As illustrated in the alternative embodiment of FIG. 5B, the dissecting punch 300b includes a dissecting distal edge 332b that is substantially flat, e.g., that forms an annulus circumventing the distal end of the lumen 324b. The annular dissecting distal edge 332b should be sized to be capable of dissecting a follicular unit when rotated at a sufficiently high speed without causing significant trauma in the area immediately surrounding the follicular unit. In one characterization, the annulus has an outer diameter of not greater than about 1.4 mm, such as not greater than about 1.2 mm. The inner diameter of the annulus may be at least about 0.1 mm, such as at least about 0.7 mm, and not greater than about 1.2 mm, such as not greater than about 1.0 mm, or not greater than about 0.9 mm. In another characterization, the thickness of the annulus (i.e., the difference between the outer diameter and the inner diameter) may be at least about 0.2 mm and may be not greater than about 0.4 mm. Other dimensions disclosed above with respect to the embodiment of dissecting punch 300 in FIG. 5A may also be applicable to the dissecting punch 300b.

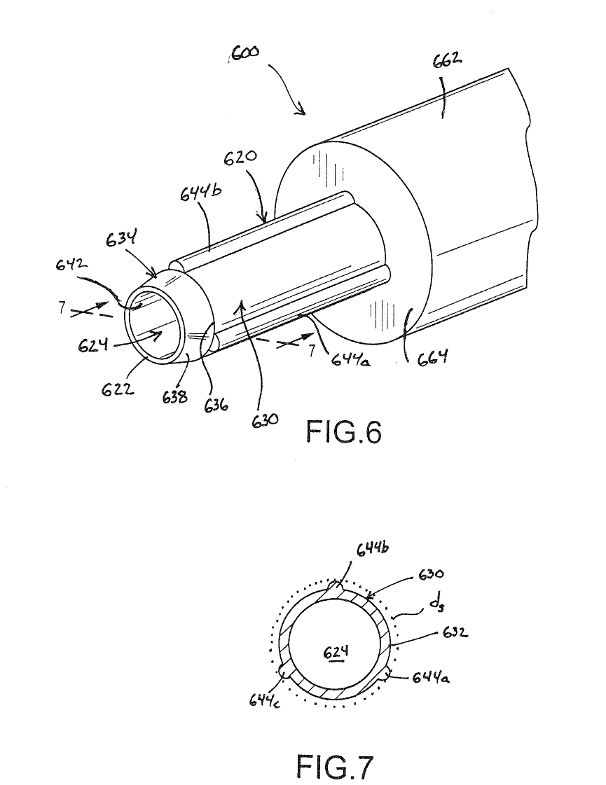

As is discussed above, other configurations of a dissecting punch are contemplated within the spirit and scope of the present disclosure. For example, FIGS. 6 and 7 illustrate an alternative embodiment of a dissecting punch 600. The dissecting punch 600 also includes a dissecting punch body 620 having a distal dissecting edge 622 and a lumen 624 disposed through at least a portion of the dissecting punch body 620. The dissecting punch body 620 includes a dissecting shaft segment 630 having an outer surface 632 and a dissecting tip segment 634 disposed at a distal end 636 of the dissecting shaft segment 630. The dissecting tip segment 634 includes an outer surface 638 extending from proximal the shaft segment 630 to the distal dissecting edge 622, which circumvents a distal end 642 of the lumen 624.

In the embodiment illustrated in FIGS. 6 and 7, the shaft segment outer surface 632 is substantially non-circular, and in this regard includes a plurality of longitudinally-extending projections 644a-644c (e.g., ribs) that extend along at least portion of the shaft segment outer surface 632. The projections 644a-644c have a slightly rounded configuration (e.g., semi-circular), although other configurations of projections (e.g., with an angular cross-section) are also contemplated. As illustrated in FIG. 6, the projections 644a-644c extend from the shaft segment distal end 636 to a shoulder of a shoulder block 662. Although illustrated as including three projections 644a-644c, the shaft segment outer surface 632 may comprise a single projection or any number of projections. Due to the presence of the projections 644a-644c, the outer surface 632 has a non-circular cross-section to facilitate the dissection of follicular units from the skin, and the projections 644a-644c provide the shaft segment 630 with an effective outer diameter dₛ.

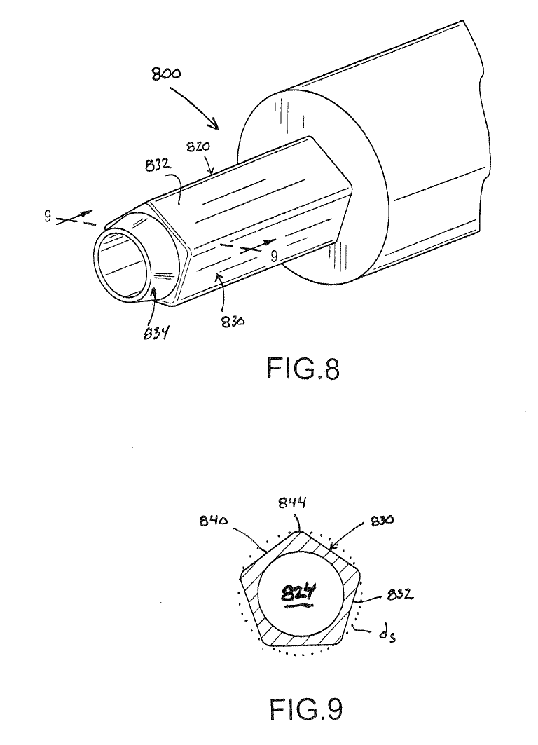

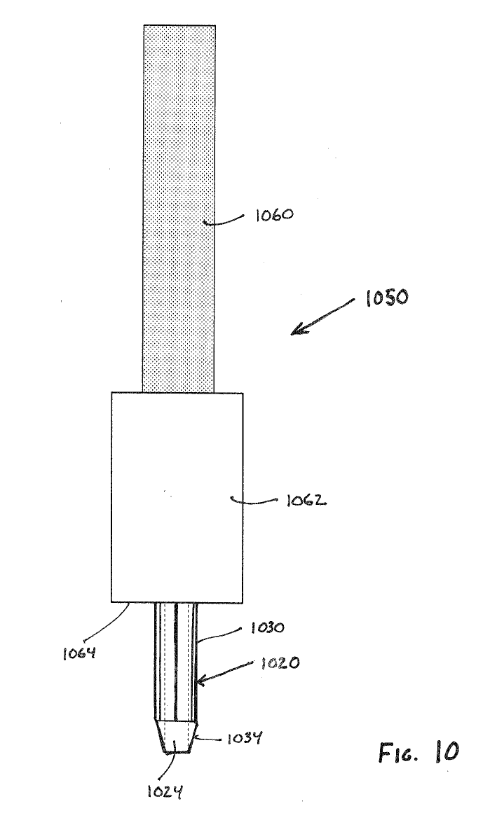

A further embodiment of a dissecting punch for dissecting follicular units is illustrated in FIGS. 8 and 9. As with the previous embodiments, the dissecting punch 800 includes a dissecting punch body 820 having a dissecting shaft segment 830 and a dissecting tip segment 834. A lumen 824 extends though at least a portion of the punch body 820. As illustrated in FIGS. 8 and 9, the shaft segment outer surface 832 defines a polygonal cross-section, in this embodiment a pentagonal cross-section. It will be appreciated that other polygonal cross-sections may also be utilized, such as a triangular cross-section or a square cross-section. Such cross-sections may also be characterized as having an outer surface 832 that includes a plurality of distinct and substantially planar surfaces (e.g., planar surface 840) that intersect adjacent planar surfaces at corners (e.g., corner 844) around the circumference of the outer surface 832.

The dissecting punches disclosed herein may be implemented in a variety of configurations. For example, the dissecting punches may be configured to be utilized in a manual fashion (e.g., non-mechanized), such as by disposing the dissecting punch body at the end of a handle that is adapted to be gripped by the user (e.g., by a surgeon). See, for example, FIG. 7 of U.S. Pat. No. 8,211,117, which is discussed above. In another example, the dissecting punch may be operated mechanically, such as by operatively coupling a rotation mechanism (e.g., an electric motor) to the dissecting punch body to rotate the dissecting punch body mechanically.

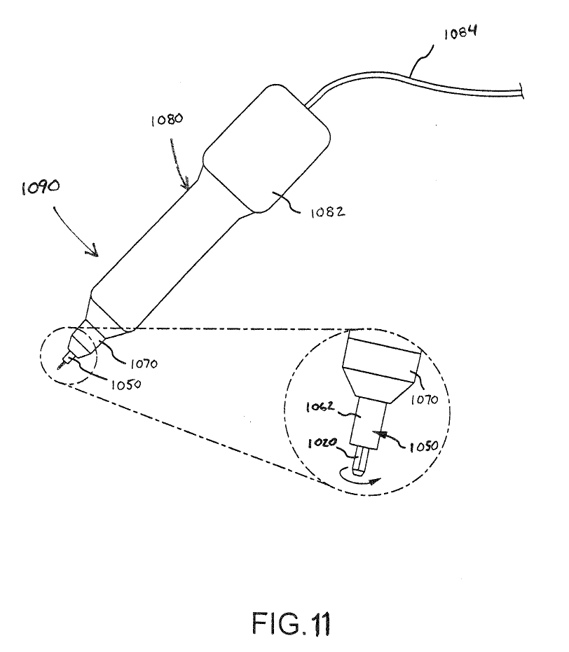

For example, FIG. 10 illustrates a dissecting punch 1050 that includes a dissecting punch body 1020 that is similar in configuration to the dissecting punch body illustrated in FIGS. 3-5A. The dissecting punch body 1020 is operatively attached to a gripping shaft 1060 that is configured to be inserted into a chuck (FIG. 11) or similar clamping device to operatively secure the dissecting punch body 1020 to a drill or similar device. For example, the surface of the gripping shaft 1060 may be serrated to facilitate the grip of the chuck on the shaft 1060. The dissecting tool assembly 1050 also includes a shoulder block 1062 disposed between the punch body 1020 and the gripping shaft 1060 and having a diameter that is larger than the diameter of the gripping shaft 1060.

As is noted, the dissecting punch 1050 may be operatively attached to a rotation mechanism such as a belt drive, an electric drill or similar device, such as by mechanically coupling the gripping shaft 1060 to the device. In this regard, FIG. 11 illustrates a tool 1090 that includes a dissecting punch 1050 that is operatively attached to a drill 1080 to enable the drill 1080 to rapidly rotate the dissecting punch 1050, e.g., at a controlled rotational speed. The drill 1080 includes a drill body 1082 that may be adapted to be easily gripped and manipulated by an operator. The drill body 1082 may also house a motor (e.g., an electric motor) for rotating the tool dissecting punch 1050 (e.g., rotating the dissecting punch body 1020) about an elongate axis of the punch body 1020. For example, the drill body 1082 may house an electric motor that is operatively coupled to a drill chuck 1070 that secures the dissecting punch body 1020 to the drill 1080. A power cord 1084 may provide power to the electric motor and may also provide signals to the electric motor, such as from a control unit, for example to control the rotational speed and/or torque of the rotating dissecting punch body 1020. However, it will be appreciated that a power supply may also be self-contained within the drill body 1082, such as by using primary or rechargeable batteries, for example. Further, a portion of the rotation mechanism may be located wholly or partially separate from the drill body 1082, such as where the dissecting punch body 1020 is rotated using an external motor that is operatively connected to the drill 1080 through a drive belt or similar external mechanism.

The dissecting punch body 1020 is disposed at a distal end of the drill body 1082 and may be generally aligned with a longitudinal axis of the drill body 1082 to enable an operator to easily grip the drill body 1082 and align the dissecting punch body 1020 (e.g., the lumen) over a follicular unit. As is noted above, the dissecting punch 1050 may be removably attached to the drill 1080 using a chuck 1070 or similar clamping mechanism that is adapted to securely retain the dissecting punch 1050. In this manner, the punch 1050 may be easily removed from the drill 1080 and replaced when needed. The punch 1050 may be autoclavable (e.g., fabricated from a metal such as stainless steel) for detachment and re-use of the punch 1050. The drill 1080 may be configured to rotate the dissecting punch body 1020 at a rotational speed of at least about 100 rpm, at least about 200 rpm, at least about 500 rpm or higher. In one particular characterization, the drill 1080 is configured to rotate the dissecting punch body at a rotational speed of at least about 2000 rpm, such as at least about 3000 rpm, at least about 3500 rpm, at least about 4000 rpm, at least about 5000 rpm or higher, such as up to a speed of about 30,000 rpm.

During the dissection of a follicular unit from the donor area of a patient, it may be desirable to rotate the dissecting punch body 1020 at a first rotational speed that is sufficiently high to score the epidermis layer of the skin, and then at a second rotational speed that is lower than the first rotational speed, to dissect (separate) the follicular unit from the surrounding fatty tissue while decreasing the probability of transecting a follicular unit with the dissecting punch 1020. In this regard, the drill 1080 may be configured to rotate the dissecting punch 1020 at two or more rotational speeds, such as over a range of rotational speeds.

For example, the rotational speed of the dissecting punch 1050 may be adjusted manually by an operator or may be adjusted in a self-regulated manner, such as by controlling the torque of the rotating dissecting punch 1050. A high torque will tend to maintain a constant or near-constant rotational speed as the dissecting punch by 1020 is moved through the dermis layer and into the fatty tissue layer of the skin. However, the application of a relatively low torque may enable the rotational speed of the dissecting punch 1050 to self-regulate and to decrease as the dissecting punch body 1020 moves through the skin layers. That is, at a sufficiently low torque, the friction between the dissecting punch body 1020 and the surrounding skin layers may cause the rotational speed to lower from a first rotational speed that is initially encountered when the dissecting punch body 1020 first contacts the skin to a lower second rotational speed as the dissecting punch 1020 moves through the dermis layer and into the fatty tissue layer. Although described above as including a single dissecting punch, e.g., a blunt dissecting punch, it is contemplated that the dissecting punch disclosed herein may also be utilized in conjunction with a sharp scoring punch, such as in the manner described in U.S. Pat. No. 8,211,117. Thus, a dissecting tool may include both a sharp scoring punch and a blunt dissecting punch as described above. As is described with respect to FIGS. 1A to 1F, the sharp scoring punch may be used to score the skin (e.g., create a shallow incision) and the dissecting punch (e.g., a blunt dissecting punch) may be used to dissect the follicular unit from the surrounding tissue in the dermis and fatty layer of the skin.

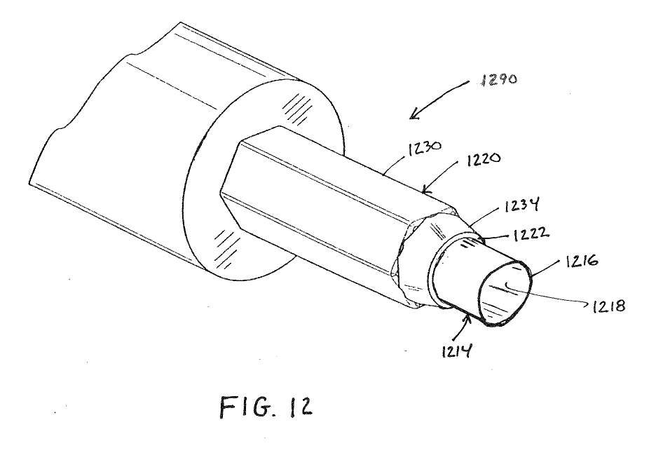

One embodiment of such a tool is illustrated in FIG. 12. In the embodiment illustrated in FIG. 12, the tool 1290 includes a dissecting punch body 1220 substantially as described above with respect to FIGS. 3 to 5. That is, the dissecting punch body 1220 includes a dissecting shaft segment 1230 and a dissecting tip segment 1234 disposed at a distal end of the shaft segment 1130. In the embodiment illustrated in FIG. 12, a sharp scoring punch 1214 is disposed within a lumen of the dissecting punch body 1220. The sharp scoring punch 1214 includes a sharp cutting edge 1216 that circumvents a sharp scoring punch lumen 1218 that extends through at least a portion of the sharp scoring punch 1214. The sharp scoring punch 1214 may be retractable within the lumen of the dissecting punch body 1220. Thus, during use, the sharp scoring punch 1214 may be utilized to score the skin surrounding the follicular unit. The sharp scoring punch 1214 may then be retracted within the dissecting punch body 1220. Thereafter, the dissecting punch body 1220 may be inserted into the scored incision formed by the sharp scoring punch 1214, while being rotated, to dissect the follicular unit from the surrounding tissue.

The embodiments disclosed herein may be implemented and utilized in a variety of configurations and methods. For example, a method for the dissection of a follicular unit may include placing a dissecting punch body having a lumen over the follicular unit and rotating the dissecting punch body while moving (e.g., inserting) the rotating dissecting punch body into the skin. The dissecting punch body should be placed over the follicular unit such that the follicular unit is disposed within the lumen when the punch body is moved into the skin. As is disclosed above, the dissecting punch body includes a dissecting shaft segment, the shaft segment having an outer surface defining a non-circular (e.g., polygonal) cross-section and a dissecting tip segment having an outer surface extending from the shaft segment to a distal dissecting edge circumventing a distal end of the lumen.

The dissecting punch body may be utilized to cut through the epidermis and score the dermis if the dissecting punch body is rotated at a sufficiently high rotational speed. Alternatively, the operator (e.g., a surgeon) may utilize a sharp scoring punch (e.g., as described with respect to FIGS. 1A to 1F and/or FIG. 12) to first score the skin (e.g., cut through the epidermis and into the dermis) before moving the rotating dissecting punch body further into the skin (e.g., through the scored incision and into the fatty layer) to dissect the follicular unit.

In one characterization, the dissecting punch body is rotated at a relatively high rotational speed during the dissection of the follicular units, e.g., during insertion of the dissecting punch body into the skin. For example, the dissecting punch body may be rotated at a rotational speed of at least about 2000 rpm, such as at least about 3000 rpm, at least about 3500 rpm and even at least about 4000 rpm. It has been unexpectedly found that the use of a dissecting punch body as disclosed herein enables the use of such relatively high rotational speeds without significantly increasing the incidence of transection of the follicular units (e.g., of the follicles), e.g., as compared to the use of a dissecting punch having a circular shaft at high rotational speeds. Furthermore, the use of such high rotational speeds may advantageously increase the rate at which follicular units may be dissected. That is, the time to carry out a procedure on a patient consisting of many individual dissections may be decreased. Such a method may be practiced with a single punch, e.g., without a sharp scoring punch.

The method may be carried out manually or using a computer-controlled (e.g., robotic) system. For example, manual methods may include the use of a hand-held device for dissection, either with or without a powered mechanism (e.g., an electric motor) for rotation of the dissecting punch and/or a scoring punch. Thus, in one characterization, the method may include the use of a powered drill (FIG. 11) that is manually manipulated (e.g., oriented) by the operator and that includes only a single dissecting punch body (e.g., without a sharp scoring punch) for the dissection of follicular units. Optionally, the rotational speed and/or torque of the dissecting punch body may be controlled by the operator, such as through the use of control knobs, foot pedals or the like. After dissection, the follicular unit may be extracted from the skin (e.g., using forceps or a vacuum) to form a follicular unit graft.

As noted, the method may also be carried out using a computer-controlled system, such as where the dissecting punch body and/or a scoring punch are affixed to a mechanized arm that is oriented to a follicular unit during dissection using computerized controls. Examples of such computer-controlled systems are disclosed in U.S. Patent Publication No. 2007/0106306 by Bodduluri et al. and U.S. Patent Publication No. 2012/0116417 by Bodduluri et al., each of which is incorporated herein by reference in its entirety. In one characterization, a single blunt dissecting punch (e.g., a dissecting punch body) is disposed on a computer-controlled robotic arm and a rotation mechanism is operatively attached to the blunt dissecting punch. In another characterization, a sharp scoring punch and a blunt dissecting punch body are disposed on a computer-controlled robotic arm, such as where the sharp scoring punch is disposed within a lumen of the blunt dissecting punch body (see FIG. 12).

### Examples

The use of tools including the dissecting punch bodies described herein for dissection may advantageously reduce the transection rate of follicular units as compared to known devices and methods. Further, the dissection rate may be decreased even when the rotational speed of the dissecting punch is increased. To demonstrate the efficacy of such tools, a trial is conducted to compare transection rates between tools described herein and known tools.

A trial is conducted involving a total of 25 patients. In a first group of 19 patients, follicular units are dissected using a mechanized dissecting tool. The mechanized dissecting tool includes a dissecting punch body operatively affixed to a drill body. The dissecting punch body comprises a dissecting distal tip and a shaft segment, where the shaft segment is circular, e.g., as illustrated and described with respect to FIGS. 2A to 2F above. During dissection of the follicular units, the dissecting drill tip is rotated at a rotational speed of about 2000 rpm. A total of 5423 dissections are performed and the transection rate is about 11.2%, i.e., 11.2% of the hair follicles were transected.

In a second group of 6 patients, follicular units are dissected using a similar mechanized dissecting tool that also includes a dissecting punch body operatively affixed to a drill body. The dissecting punch body comprises a dissecting distal tip and a shaft segment that has a hexagonal cross-section, e.g., substantially as described above with respect to FIGS. 3 to 5A. During dissection of the follicular units, the dissecting punch body is rotated at a rotational speed of about 4000 rpm. A total of 8164 dissections are performed and the transection rate is about 2.55%, i.e., 2.55% of the hair follicles are transected.

While various embodiments of the present invention have been described in detail, it is apparent that modifications and adaptations of those embodiments will occur to those skilled in the art. However, it is to be expressly understood that such modifications and adaptations are within the spirit and scope of the present invention.

1-48. (canceled)

49. A method for the dissection of a follicular unit from the skin of a donor, comprising the steps of:
placing a dissecting punch body having a lumen over a follicular unit;
rotating the dissecting punch body; and
moving the rotating dissecting punch body into the skin whereby the follicular unit is disposed within the lumen;
wherein the dissecting punch body comprises;
a dissecting shaft segment, the shaft segment comprising an outer surface defining a non-circular cross-section; and
a dissecting tip segment disposed at a distal end of the shaft segment, the dissecting tip segment comprising an outer surface extending from the shaft segment to a distal dissecting edge circumventing a distal end of the lumen.

50. The method recited in claim 49, further comprising the step of scoring the skin over the follicular unit with a sharp scoring punch before moving the rotating dissecting punch body into the skin.

51. The method recited in claim 50, wherein the sharp scoring punch is retractable within the lumen of the dissecting punch body.

52. The method recited in claim 49, wherein the dissecting punch body is rotated at a rotational speed of at least about 3000 rpm during the moving step.

53. The method recited in claim 49, wherein the dissecting punch body is rotated at a rotational speed of at least about 3500 rpm during the moving step.

54. The method recited in claim 49, wherein the distal dissecting edge is substantially circular.

55. The method recited in claim 54, wherein the distal dissecting edge has an internal diameter of at least about 0.5 mm and not greater than about 2.0 mm.

56. The method recited in claim 49, wherein the dissecting tip segment has a length of at least about 0.5 mm and not greater than about 2.0 mm.

57. The method recited in claim 49, wherein a diameter of the dissecting tip segment outer surface is tapered inwardly from proximal the shaft segment to the distal dissecting edge to facilitate insertion of the tip segment into the skin surface.

58. The method recited in claim 49, wherein the shaft segment outer surface comprises at least a first longitudinally-extending projection.

59. The method recited in claim 58, wherein the shaft segment outer surface comprises a plurality of longitudinally-extending projections.

60. The method recited in claim 49, wherein the shaft segment outer surface defines a polygonal cross-section.

61. The method recited in claim 60, wherein the shaft segment outer surface defines a hexagonal cross-section.

62. The method recited in claim 49, wherein the shaft segment has a length of at least about 2.0 mm and not greater than about 6.0 mm.

63. The method recited in claim 49, wherein the shaft segment has an effective outer diameter of at least about 1.0 mm and not greater than about 1.7 mm.

64. The method recited in claim 49, wherein the lumen extends through the shaft segment.

65. The method recited in claim 49, further comprising a shoulder at a proximal end of the shaft segment, the shoulder having an outer diameter greater than an effective outer diameter of the shaft segment.

66. The method recited in claim 49, wherein the distal dissecting edge has a radius of curvature of at least about 0.025 mm.

67. The method recited in claim 49, wherein the distal dissecting edge is substantially flat.

68. A method for the dissection of a follicular unit from the skin of a donor, comprising the steps of:
placing a dissecting punch body having a lumen over a follicular unit;
rotating the dissecting punch body at a rotational speed of at least about 3000 rpm; and
moving the rotating dissecting punch body into the skin whereby the follicular unit is disposed within the lumen;
wherein the dissecting punch body comprises;
a dissecting shaft segment, the shaft segment comprising an outer surface defining a polygonal cross-section and having an effective outer diameter of at least about 1.0 mm and not greater than about 1.7 mm; and
a dissecting tip segment disposed at a distal end of the shaft segment, the dissecting tip segment comprising an outer surface extending from the shaft segment to a substantially circular distal dissecting edge circumventing a distal end of the lumen, wherein at least a portion of the lumen extends through the dissecting tip segment and is substantially circular.
